(19) United States
(12) Patent Application Publication
(10) Pub. No.: US 2019/0328957 A1
(43) Pub. Date: Oct. 31, 2019

(54) OXYGEN OZONE REGENERATIVE THERAPIES WITH ALL INFORMATION ON TREATED PATIENTS; INCLUDED ORGAN TRANSPLANT PATIENTS AND NEW LAB DATA WITH KIDNEY TISSUE CONTAINING STEM CELLS PICTURED GROSSLY IN PATIENT URINE AND STEM AND PROGENITOR CELLS OR CD 34 POSITIVE CELLS PICTURED IN CLUMPS IN URINE

(71) Applicants: Priya Visweswaran Balakrishnan, Houston, TX (US); Sambhu Mahadeva Balakrishnan, Houston, TX (US)

(72) Inventors: Priya Visweswaran Balakrishnan, Houston, TX (US); Sambhu Mahadeva Balakrishnan, Houston, TX (US)

(21) Appl. No.: 16/459,535

(22) Filed: Jul. 1, 2019

Related U.S. Application Data
(63) Continuation-in-part of application No. 15/413,410, filed on Jan. 23, 2017, now Pat. No. 10,335,538.
Application No. 15/413,410 is a continuation-in-part of application No. 15/373,453, filed on Dec. 8, 2016.
(60) Provisional application No. 62/281,919, filed on Jan. 22, 2016.
(60) Provisional application No. 62/281,616, filed on Jan. 21, 2016.
(60) Provisional application No. 62/264,522, filed on Dec. 8, 2015.

(51) Int. Cl. A61M 5/14; A61B 17/132; A61M 5/158; A61M 5/31

(52) U.S. Cl. CPC A61M 5/1407; A61B 17/132; A61M 2005/006; A61M 5/31; A61M 5/158

(57) ABSTRACT

A treatment method to cure diseases within a patient by administering intravenous oxygen-ozone mixture (Oxygen Ozone Regenerative Therapies, OORT). Specifically, the present application discloses a method of administering an intravenous oxygen-ozone mixture (Oxygen Ozone Regenerative Therapies, OORT). The method comprises the steps of: (A) providing a syringe, an infusion device and a tourniquet; (B) identifying an accessible peripheral vein on an upper extremity of a user; (C) preparing a volumetric dosage of an oxygen-ozone mixture (Oxygen Ozone Regenerative Therapies, OORT) with the syringe; (D) applying the tourniquet to a cannulation area on the upper extremity and inserting the infusion device into the accessible peripheral vein; and (E) transferring the volumetric dosage of the oxygen-ozone mixture (Oxygen Ozone Regenerative Therapies, OORT) from the syringe through the infusion device and into the accessible peripheral vein at a specified infusion rate by releasing the tourniquet from the cannulation area after a witnessed flash of blood

### FIELD OF THE INVENTION

The present invention generally relates to a method of using an ozone-oxygen mixture, we are calling Oxygen Ozone Regenerative Therapies or OORT, to treat various diseases. More specifically, the present invention intravenously administers the ozone-oxygen mixture to a patient through the use of a syringe and an infusion device, by reactivating our proposed (first time in western medicine), putative Regenerative Organ System.

### BACKGROUND OF THE INVENTION

The present invention concerns a method of administration of oxygen-ozone mixture (Oxygen Ozone Regenerative Therapies, OORT) to cure disease. Usages of the method disclosed in the present invention include but are not limited to treating conditions such as Chronic Kidney Disease, End Stage Renal Disease, Diabetes, Heart Disease (Coronary Artery Disease, Congestive Heart Failure), Neurological diseases such as neuropathy and strokes and degenerative diseases, including dementia, Parkinsons's disease, ALS and MS, Hypertension, High Cholesterol, COPD, Atrial Fibrillation, Osteoporosis, Osteoarthritis, Asthma, Depression, Hepatitides, and Lyme Disease, genital herpes and HIV. It is also conceivable that the present invention may be used to treat every infectious disease including Ebola, inflammatory and autoimmune diseases, chronic fatigue, and like diseases. The present invention may be used in cancer with variable results.

To summarize the differences, the present application is for a human clinical procedure, given to 60 patients safely and highly effectively, in a pilot clinical trial conducted in Houston in 2015, given for therapeutic purposes, which yields improvements in the treatments of multiple chronic medical illnesses, including and especially Chronic Kidney Disease (CKD) and End Stage Renal Disease (ESRD), heart disease, degenerative neurological diseases such as multiple sclerosis and others, diabetes both Types I and II, and other degenerative diseases such as osteoarthritis, depression, hypertension, early and easy fatiguability, fibromyalgia, stroke and multiple other chronic medical illnesses.

The present application discloses a method of administering an intravenous oxygen-ozone mixture (Oxygen Ozone Regenerative Therapies, OORT). The method comprises the steps of: (A) providing a syringe, an infusion device and a tourniquet; (B) identifying an accessible peripheral vein on an upper extremity of a user; (C) preparing a volumetric dosage of an oxygen-ozone mixture (Oxygen Ozone Regenerative Therapies, OORT) with the syringe; (D) applying the tourniquet to a cannulation area on the upper extremity and inserting the infusion device into the accessible peripheral vein; and (E) transferring the volumetric dosage of the oxygen-ozone mixture (Oxygen Ozone Regenerative Therapies, OORT) from the syringe through the infusion device and into the accessible peripheral vein at a specified infusion rate by releasing the tourniquet from the cannulation area after a witnessed flash of blood. In other words, the present application transfers the volumetric dosage of the oxygen-ozone mixture (Oxygen Ozone Regenerative Therapies, OORT) from the syringe through the infusion device and into the accessible peripheral vein with specified concentrations and volumes, with and without obstacles to administration, locates the vein, and makes it prominent enough to make it accessible to receive the treatment.

A patient was given methylcobalamin (cyanocobalamin, if the patient does not have chronic kidney disease, or hydroxocobalamin may also be substituted instead.) intramuscularly as well, as needed, for which a sliding scale is utilized, depending upon the patient's bodily habitus, stress levels, prior deficiency status and disease status, age and other co-morbid conditions. On average patients run between 5 mg and 30 mg in weekly in divided doses between 3 times a week to daily, per the sliding scale. This is not to say that lower doses will not have a beneficial effect. This is just to add this to the protocol, and to demarcate that this is the dosage range within which optimal results are seen. Other steps have been added for individual patients. That work to enhance or optimize this protocol, including a particular kind of Vitamin and supplements mixture including living probiotics, and rudraksha beads usage and yoga therapy and other interventions such as IV supplements and vitamins and hormone supplementation, as the case may require. These are supplementary steps and require more clinical definition before adding specifically to a patent that in its most core form, applies to all diseases and all conditions, and all patients.

As mentioned previously, the Applicant had to undertake a pilot clinical trial in 60 human beings, with different degrees of chronic kidney disease, and other chronic medical illnesses, and observe minutely, changes in their blood and urine tests with different concentrations of the various oxygen containing gases, in mixture. The Applicant had to make extensive clinical observations (and correlate them with possible changes in Regenerative Organ System functionality improvement, based on observation of improvement in different organs and organ systems with the treatment, and hypothesize or postulate, particularly what stem cells or what progenitor cells could have increased in number and functionality—which has never been done before clinically—a clinical correlation based on regenerative changes in the human body) over the course of multiple treatments given to each patient, at different concentration compositions to study minimum effective dose in multiple chronic medical diseases, from a very small starting dose, in order to first not harm the patient, unlike the very high a dose utilized by other treatments, in one single dog with leukemia.

The Applicant's clinical observations of improvements in patients with significant chronic medical illnesses, such as improvement in creatinine and proteinuria in patients with Chronic Kidney Disease (in one patient creatinine reduced enough to delay dialysis by two years and in another patient, creatinine reduced from 1.3 mg/dl to 0.9 mg/dl with just 20 sessions), or improvement in kt/v from 0.9 to 1.58 in a patient with End Stage Renal Disease (with 30 sessions of treatment), while already on dialysis and previously not making any urine (strongly suggesting increases in circulating numbers of hematopoietic stem and progenitor cells, hemangioblasts and endothelial and nephron progenitor cells, who now started making copious quantities of urine, and very specific improvements in patients with Multiple Sclerosis, revealing an increase in growth in patient's muscles and strength and caliber, and the size and number of veins from thready and few, to many jungle vine like structures over a few short treatments, suggested to Applicant that the protocol she was using, of which this she believes is the keystone step, which are both results of reactivation of the Regenerative Organ System in the human body, which includes reactivating neurons supplying the bone marrow and the kidney (lead organ in the Regenerative Organ System) which release stem and progenitor cells from these locations, particularly the kidney even, which is a previously undescribed (beyond a certain embryonal stage) potential adult site of regenerative activity within the human body (which our keystone step is activating), which result in increases in stem and progenitor cell counts, specifically to the clinical disease or scenario, none of which have been enumerated by prior art.

Applicant as an expert in this art, feels that while there is an intuitive approach to all this on her part, about this protocol or treatment of the present application, that this is not intuitive to anyone else who is an expert in the field, or they will have proposed it, as the significance of what is being described, is monumental and Nobel-prize worthy. And the effect of this gaseous mixture on the human body, is ancient, ancestral and astrobiological, from when life entered planet earth atmospherically, and has been the transmission of a searing memory of exposure to various forms of oxygen in abundance, intergenerationally, across all earth life forms, and will be the subject of many, many Nobel prizes in the future. To one, it seems that there is likely to be nothing intuitive or practically obvious about Applicant's observations, for these reasons.

### BRIEF DESCRIPTION OF THE DRAWINGS

FIG. 1 is a flowchart illustrating an over process for the method of the present invention.

FIG. 2 is a flowchart illustrating a sub-process for implementing a specific embodiment of the medicinal-administration system.

FIG. 3 is a flowchart illustrating a sub-process to more efficiently administer the intravenous oxygen-ozone mixture (Oxygen Ozone Regenerative Therapies, OORT).

FIG. 4 is a flowchart illustrating another sub-process to more efficiently administer the intravenous oxygen-ozone mixture (Oxygen Ozone Regenerative Therapies, OORT).

FIG. 5 is a flowchart illustrating another sub-process to more efficiently administer the intravenous oxygen-ozone mixture (Oxygen Ozone Regenerative Therapies, OORT).

FIG. 6 is a flowchart illustrating a sub-process to more effectively execute the treatment sessions.

FIG. 7 is a flowchart illustrating another sub-process to more effectively execute the treatment sessions.

FIG. 8 is a flowchart illustrating another sub-process to more effectively execute the treatment sessions.

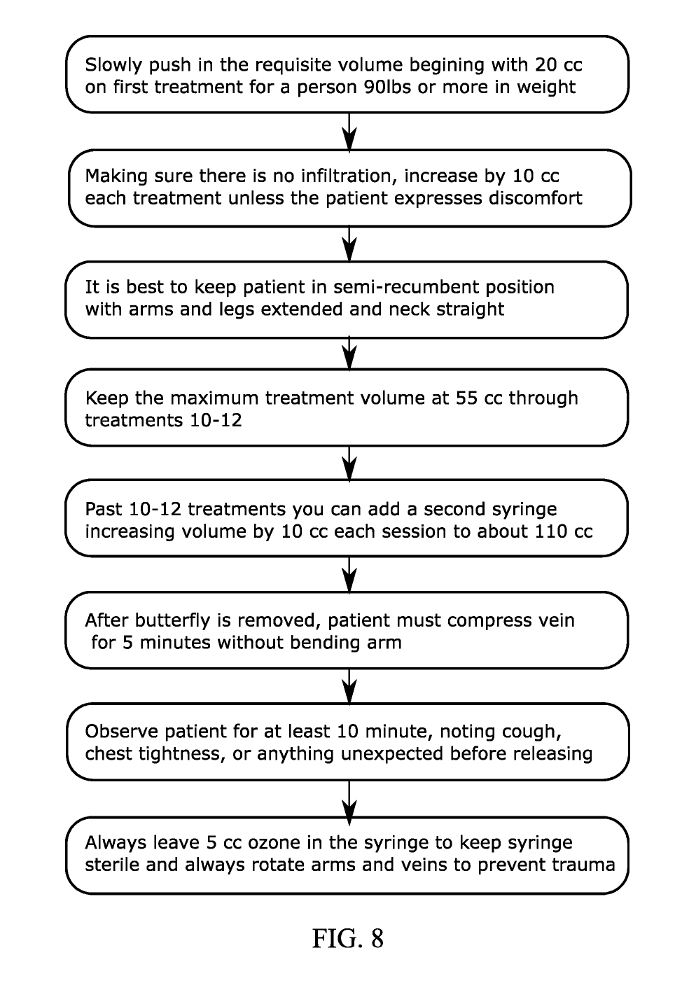

### BRIEF SUMMARY OF INVENTION

A method of administering intravenous oxygen-ozone mixture (Oxygen Ozone Regenerative Therapies, OORT) comprises the steps of providing a syringe, an infusion device and a tourniquet, identifying an accessible peripheral vein on an upper extremity of a patient, preparing a volumetric dosage of an oxygen-ozone mixture (Oxygen Ozone Regenerative Therapies, OORT) with the syringe, applying the tourniquet to a cannulation area on the upper extremity and inserting the infusion device into the accessible peripheral vein and transferring the volumetric dosage of the oxygen-ozone mixture (Oxygen Ozone Regenerative Therapies, OORT) from the syringe through the infusion device and into the accessible peripheral vein at a specified infusion rate by releasing the tourniquet from the cannulation area after a witnessed flash of blood.

This procedure is undertaken to reactivate a latent or dormant what we propose is a putative and yet highly likely to exist, Regenerative Organ System within the human body, by triggering a physiological signaling within the entire neuronal network of the body of the excess availability of oxygen, thereby activating neurons in the bone marrow (CEO organ of the Regenerative Organ System) and Kidney (brain or lead organ of this putative Regenerative Organ System), leading to an organism-wide reactivation of this Regenerative Organ System and the human body's own capacity to heal itself, which is very different from what has been previously proposed as the mechanism of action of a different combination of similar gases (0.14-1.7 ml/kg of human body weight), for different durations (30 treatments to 6-7 months for ours), different proposed mechanisms of action (activating normal hematopoietic cells to replaces leukemia cells in dogs, versus activating whole body neuronal network to reactivate latent and partially dormant Regenerative Organ System in ours via an ancient, ancestral and astrobiological trans-species signal about the abundant availability of oxygen, resulting in greater circulating numbers of hematopoietic stem and progenitor cells, endothelial progenitor cells, hemangioblasts and nephron progenitor cells and other stem and progenitor cells (both from the effector bone marrow organ and from the kidney), as evidenced clinically by increased muscle mass, hair growth, size and number and caliber of veins and other blood vessels, vastly improving kidney function chemistry parameters, vastly better cholesterol and lipids in blood work, reduced or eliminated proteinuria, better hemoglobin and white blood cell counts seen in immunosuppressed patients with autoimmune disease and many others, as seen in 60 patients enrolled in our pilot clinical trial expressly to determine efficacy of treatment utilizing this gaseous mixture in chronic kidney disease and other chronic medical illnesses, as well as therapeutic dosage window and not to exceed dose and lowest dose at which therapeutic effect is observed).

We would respectfully and humbly submit to the USPTO that this is original work undertaken after studying literature that has been published and others' work, and required a considerable degree of non-intuitive, non-standard, non-extrapolative, more intuitive and insightful work on our part, than anything that could be scaled up and down an experimental curve like a sliding scale, which our work has not been, or logically computed based on prior art or any other prior published paper.

### OBJECT OF THE INVENTION

It is an object of the present invention, to overcome deficiencies in prior art of the treatment of human beings suffering from chronic medical illnesses, such as chronic kidney disease, end stage renal disease, diabetes, osteoarthritis, multiple sclerosis, chronic fatigue syndrome, chronic pain related to injuries etc.

The Applicant is describing the present invention as it relates to the reversing of chronic kidney disease and end stage renal disease, without the other multiple steps provided by our institution, around this keystone step, in order to reverse kidney failure and all other chronic medical diseases.

### Nature of Invention

This keystone step lies in the intravenous administration of a very specific concentration of a mixture of oxygen and ozone gases, in a very specific amount, in escalating dosages as observed works and described in the present application, with attention to possible contraindications, and potential side effects within human recipients, and with attention to what constitutes sufficient administration for the treatment of the chronic medical illness, clinically, and using this therapeutic administration of this very particular and specific gaseous mixture to optimally reactivate what we are reporting to be the Regenerative Organ System in the body and thereby reverse all chronic medical illnesses, effectively enough that patients may drastically reduce medication use, drastically improve disability and inability to work, and drastically improve healing from all acute illnesses and help regain youthfulness, disease-free state and longevity (as measured by multiple markers—telomere length, telomerase activity, return to normal gene expression (with the synthesis of adequate quantities of properly folded and functioning proteins), return to normal circulating and tissue count of stem and progenitor cells, reduction or elimination in gene expression errors, reduction or elimination in inefficient cellular communication, normally functioning proteins, as opposed to abnormally structured and low functioning proteins, balanced metabolism, healing of DNA damage and prevention of DNA damage more effectively, regaining and resumption of body energy production and machinery functionality from mal-functionality, and normal cell death utilizing normal dying and scavenging processes (without abnormal immortal processes being activate at a cellular level or abnormal inactivation of normal cellular death processes, or cell death occurring by abnormal death processes).

The state of chronic medical illness contributes to accelerated aging and more premature death, a state that costs our country almost $1 Trillion a year in CMS expenditure. Just as an example, $0.5 Trillion of that is expended on 8 million chronically ill patients with 6 or more diagnoses—all the rest is expended on another several million people with less than 6 chronic medical illnesses.
The Applicant wishes to further describe changes observed in the Applicant's patients in the Applicant's pilot trial: the Applicant treated multiple patients with multiple sclerosis, several patients with chronic kidney disease, people with type II diabetes, type I diabetes, former injuries, osteoarthritis, chronic fatigue syndrome, hepatitis c, polio survivor, HSVII, chronic infections such as chronic fungal sinusitis, fibromyalgia and other diagnoses.
The Applicant's patient with Multiple Sclerosis, who is a naturopathic doctor herself, started to stand up from her wheelchair to which she was confined, and move around with only a walker for the first time in years, her mood improved tremendously, her ankle fractured healed far more rapidly than originally predicted.
Multiple sclerosis is of course a disease of neurons in the brain, and can progress with relapses and remissions unto the end stage of the disease.
Two more patients of this patient herself, with the same disease, also did exceptionally well under our care, utilizing the ozone-based therapies the Applicant gave them.
One of them, a Palestinian former accountant in her 70s, who was formerly bedridden with flaccid useless arms and legs, who started to move her hands, forearms (flipping burgers), arms (started assisted her husband in lifting herself out of bed, utilizing a steel frame mounted over the frame of the bed by him) and to write again (first time in 10 years) and regenerated at least 10 forearm veins (making one suspect based on prior Cardiology reports correlating regeneration of coronary arteries and leg veins to numbers of circulating endothelial progenitor cells, diabetes and smoking only, in secondary cardiac prevention post-MI, and based on the Applicant's own clinical observations with a number of patients, that the numbers of hematopoietic stem and progenitor cells, and endothelial progenitor cells are hugely increased with these treatments; and therefore by extension, possibly the numbers of hemangioblasts, which are the precursor cells to both types of cells), after receiving just 30 treatments of the ozone with the Applicant.
Not only is it untrue that MS is an incurable disease, it is also untrue that neurons that die do not grow back, and it is also untrue that stem cells are no longer circulating after the age of 50. Oxygen Ozone Regenerative Therapy or OORT (which is what we shall call this) makes all of these statements untrue (The Applicant's has personally witnessed it in multiple patients with neurological diseases, whom the Applicant has treated with it).
In addition to reversing chronic kidney disease (published for the first time in the Journal of the American Society of Nephrology Abstracts Book for Kidney Week in October 2016) in more than one patient, as measured by both their serum creatinine and their estimated glomerular filtration rate, the Applicant successfully reversed end stage renal disease as well.
When this gaseous mixture is administered to diabetic patients, their insulin requirements drastically reduce as does neuropathy.
Osteoarthritic patients, especially with knee problems are significantly better without surgery, allowing free and easy mobility, after even just one dose of this therapy.
Chronic Fatigue syndrome patient missed her afternoon nap for the first time in 3 years after suffering EBV infection, after just one dose.
Fibromyalgia patients' chronic pain was hugely improved; as was irregular menstrual bleeding and pain in patients with fibroids (menstrual cycle regularized and pain disappeared).
Depression is dramatically alleviated suggesting that depression is better when circulating stem and progenitor cell counts increase exponentially, instantaneously.
Hypertension and atrial fibrillation are better.

Stroke patients can regain mobility if seen within a few months' after the stroke.

### Inventive Concept

The Applicant is describing in the present application, combination of gases, when administered thus within human beings, causes reactivation of a silenced regenerative organ system (REGENOS), and results in the release of multiple types of stem and progenitor cells, which facilitates reversal of the chronic medical illness, short-term. It is possible that this combination of stem and progenitor cells that are released by this protocol containing highly available and usable species of oxygen molecules caused by reactivation of the Regenerative Organ System, can also affect the outcome of cancer, as cancer thrives in hypoxia or low ambient tissue oxygen levels, whereas, cancer stem cells which proliferate in low oxygen environments, can be replaced or overthrown or overgrown by healthy stem and progenitor cells, which grow in high oxygen availability environments, such as is created by our procedure, something which has not been discussed or proposed as a mechanism of action by the prior art, nor is intuitive from her usage of very toxic, very, very high doses of reactive oxygen species in her prior art.

The Applicant is not utilizing a modulation of the immune system within this protocol, and however, that as in the patient described here, OORT maybe utilized carefully, in patients with organ transplant, to avoid precipitating acute rejection, as described here, and as performed previously herein.

The Applicant is not utilizing a modulation of the immune system within this protocol, the Applicant believes the protocol to administer IV combination of oxygen and ozone gases, must be considered to be carefully indicated, in any clinical situation in which the patient has a transplanted organ within their body; whereas, in our one patient with a failed kidney transplant who is currently back on hemodialysis, acute rejection of the organ has not been triggered by OORT.

The mechanism by which the Applicant believes this gaseous mixture is acting, is the triggering of endothelial cell and neuronal activation is the triggering of endothelial cell activation only at the exact instant of injection, and at the site of injection of the gaseous mixture, which is rapidly absorbed (within nanoseconds) by the blood cells by ozonation of their lipid membranes.

This application converts the anaerobic respiration within neurons of the vascular walls, a switch that might have been caused due to injury, inflammation or impenetrability of blood supply through the vascular walls to the neurons, into a full-fledged large-scale aerobic respiration, which results in the activation of all neurons including those in bone marrow, whose, electron transfer (or current) causes the generation (proliferation and activation) of stem cells and those neurons in the kidney, which causes a huge proliferation independent of the bone marrow produced stem cells, within the kidney even, which is not previously known to be an organ of regeneration, in any human life, other than embryonal (not in children or in adults). This transmission of the aerobic respiration activation signals to all neurons which are interconnected forming a sleeve along the length of all blood vessel walls, conduct the impulses conveying the excess availability of oxygen, this ancient, ancestral and astrobiological signal, to all networked neurons, all over the body, which results in the turning on of homing signals for stem cells where injury is now perceived (previously not, due to underlying neuronal inactivation or death), and reactivation of smooth and striated muscles overlying neurons everywhere (including a more rapid gut transit time and easier clearance of respiratory secretions and urine), by this potential reactivation of an entire putative and proposed by us alone and us for the first time, regenerative organ system.

This mechanism of action we have proposed based on our observations of our End Stage Renal Disease patient, who started making urine after receiving the very first dose of this therapy. When he subsequently underwent dialysis, his urine, which I'd asked him to collect in Mason Jars to show me that he was indeed making it, turned white, from clear yellow (and had solid matter in it, like tender coconut or buttermilk). Since I did not have a microscope or lab at the time, I proposed that these were stem cells and progenitor cells, including hemangioblasts, hematopoietic stem and progenitor cells, endothelial progenitor cells, that were being made post-filter de novo in the kidney for the first time ever, since the embryo stage of the patient, and that they were being shed into the urine owing to the systemic inflammation caused by being on hemodialysis for four hours (with multiple use recycled plastic capillaries within the dialysis filter), which culminated within the filtration units or glomeruli of the kidney, torching these cells and causing them to fall into the urine, to avoid the inflammatory burn, without forming adhesions between each other and differentiating into the cells that needed to form, to regenerate the kidney (40 types of cells within the kidney, which are lost during scarring and end stage renal disease). When the patient was administered a potent anti-inflammatory substance namely a form of curcumin which is absorbed without being detoxified by first-pass metabolism within the liver, and given electromagnetic therapy as a beacon to draw and retain these immature cells within the kidney and advance their maturation rapidly, the patient stopped spilling/shedding these regenerative cells in the urine, despite still being on dialysis (and continuing to still have systemic inflammation from dialysis) and the urine returned to yellow and clear, and he resumed recovery to normalcy, of his renal function, caused by the unhindered actions of these regenerative cells within the kidney itself.

The interesting thing about the metabolism of intravenous ozone-oxygen mixtures, is that the presence of the ozone component is only nanoseconds long, within which time, it combines with membrane lipids of red blood cells and white blood cells, thereby extinguishing its oxygen-abundance-signal almost instantly. However, the single neuron that has come into contact with a molecule of ozone oxygen, has already activated the entire network of neurons, which are now processing glucose via an aerobic pathway, leading to enhanced energy generation in every cell, leading to better structure and function of proteins and therefore better functionality of the cell, leading to its revivification.

This is an entirely new mechanism of action for the particular mixture that the Applicant is proposing for the very first time, and this mechanism that the Applicant is proposing is based on our clinical data and observations in human beings, and past in vitro and animal in vivo experimental data by others.

### DETAILED DESCRIPTION OF THE INVENTION

All illustrations of the drawings are for the purpose of describing selected versions of the present invention and are not intended to limit the scope of the present invention.

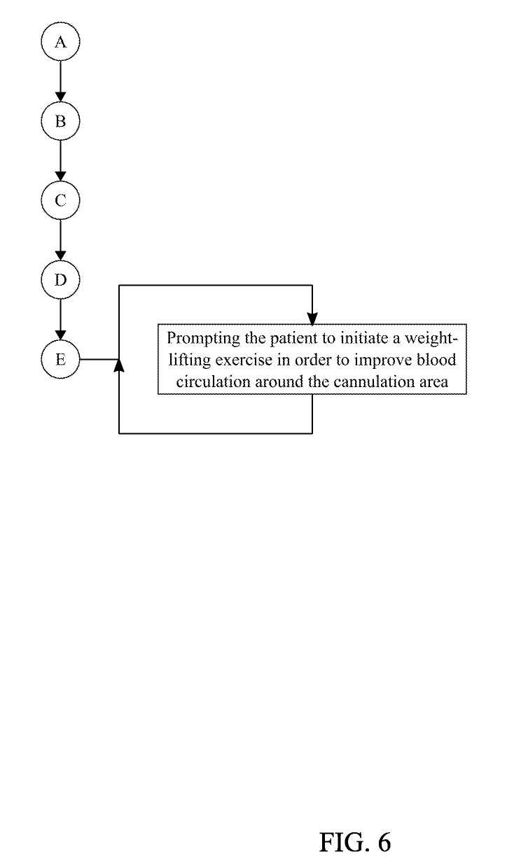

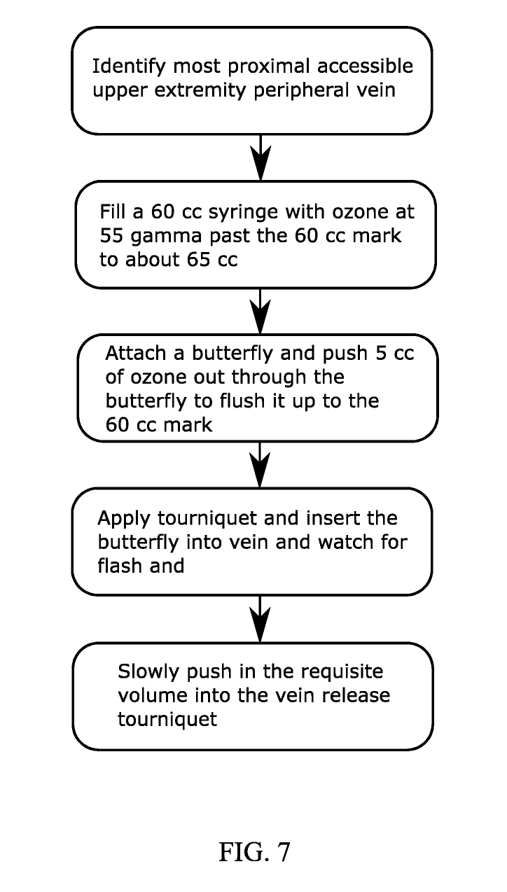

In reference to FIGS. 6 and 7, the present invention is a system and a method to cure diseases with a patient by administering intravenous oxygen-ozone mixture (Oxygen Ozone Regenerative Therapies, OORT). A proprietary machine has been designed to create this gaseous oxygen-ozone mixture (Oxygen Ozone Regenerative Therapies, OORT) with precision concentration controls and sterility. This gaseous oxygen-ozone mixture (Oxygen Ozone Regenerative Therapies, OORT) is then injected into the patient increasing doses starting with 10 to 20 milliliters depending upon the patient's weight and the patient's habitus. Common contraindications to the usage of this therapeutic mixture include recent (less than 3 months) acute myocardial infarction, pregnancy, and bleeding diathesis and Glucose-6 Phosphate Dehydrogenase enzyme absolute or acquired deficiency (gleaned by eliciting a history of sulfa drug allergy, or a blood test for the enzyme level if the patient has never had a sulfa drug). In addition, an administrator is generally provided with a syringe, an infusion device, and a tourniquet (Step A) in order to complete the overall process of the present invention. In the preferred embodiment, the syringe is configured to retain a volume of 60 mL, and the infusion device is a 27-gauge winged infusion set that is known in the relevant arts as a butterfly. The administrator should also be a licensed medical practitioner.

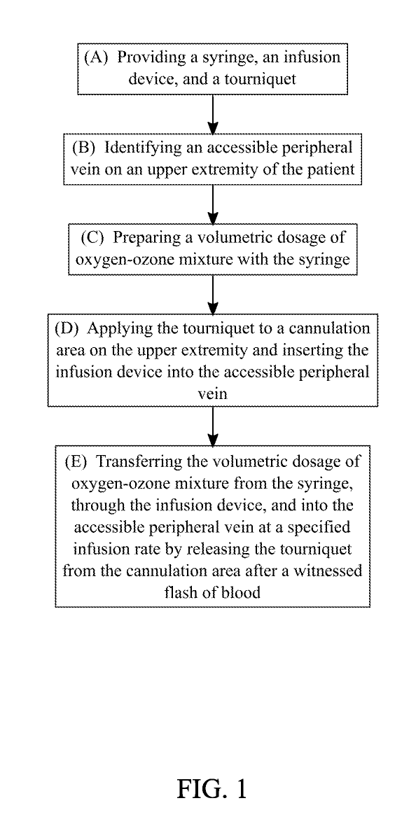

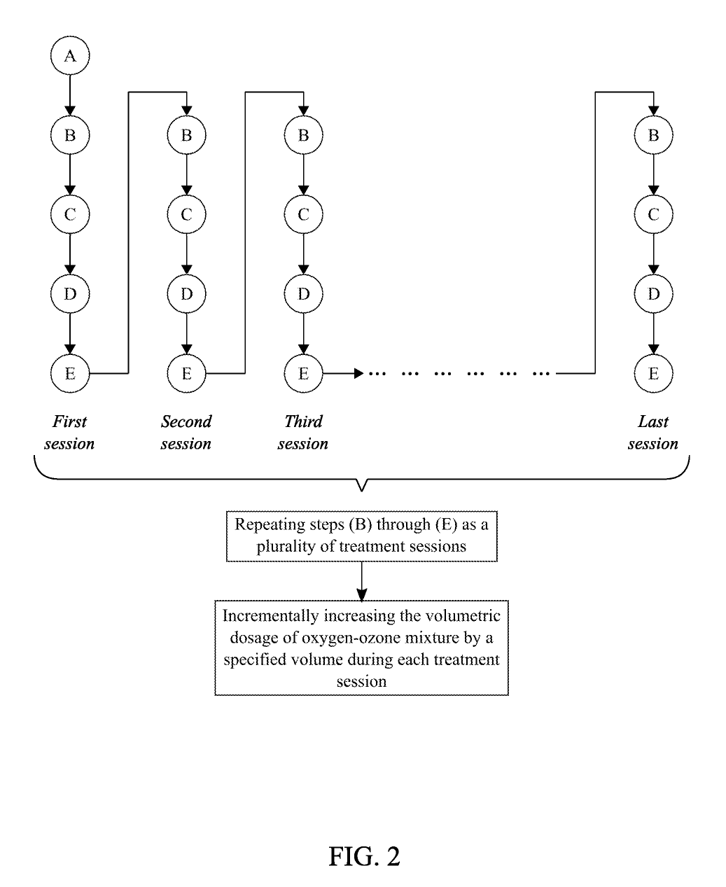

As can be seen in FIG. 1, the overall process of the present invention begins by allowing the administrator to identify an accessible peripheral vein on an upper extremity of the patient (Step B). The upper extremity is preferably the arm or the elbow of patient. The administrator then prepares a volumetric dosage of oxygen-ozone mixture (Oxygen Ozone Regenerative Therapies, OORT) with the syringe (Step C), which is used to retain and intravenously inject the volumetric dosage of oxygen-ozone mixture (Oxygen Ozone Regenerative Therapies, OORT) into the patent. In the preferred embodiment, the a 60-mL syringe is filled with ozone at 55 gamma (55 mcg/mL) past the 60-mL mark to about the 65-mL mark. Also in the preferred embodiment, the oxygen-ozone mixture (Oxygen Ozone Regenerative Therapies, OORT) is approximately composed of 96% oxygen and 4% ozone. The overall process continues as the administrator applies the tourniquet to a cannulation area on the upper extremity and subsequently inserts the infusion device into the accessible peripheral vein (Step D). The tourniquet is applied to the cannulation area in order to improve blood circulation through the veins of the patient, which in turn allows for easier execution of Step E. After the administrator witnesses a flash of blood within the cannulation area, the administrator releases the tourniquet from the cannulation area. This allows the overall process to conclude by transferring the volumetric dosage of oxygen-ozone mixture (Oxygen Ozone Regenerative Therapies, OORT) from the syringe through the infusion device, and into the accessible peripheral vein at a specified infusion rate (Step E). The volumetric dosage of oxygen-ozone mixture (Oxygen Ozone Regenerative Therapies, OORT) can then be slowly infused into the accessible peripheral vein. The preferred infusion rate is approximately 1 mL every 5 to 15 seconds, and the preferred duration for Step E should last one to two minutes. Yet, the proper infusion rate is proportionally dependent on the diametrical size of the accessible peripheral vein. Thus, the administrator can adjust the specified infusion rate to be proportionately slower if the accessible peripheral vein has a smaller diameter. The present invention also allows the overall process to be repeated between the administrator and the patient as a plurality of treatment sessions, which is shown in FIG. 2. Thus, Step B through Step E is repeated during each treatment sessions. In order for the patient to garner the most health benefits from receiving multiple treatment sessions, the administrator incrementally increases the volumetric dosage of oxygen-ozone mixture (Oxygen Ozone Regenerative Therapies, OORT) by a specified volume during each treatment session. The specified volume is a dosage escalation that is proportionately depended upon a weight, a habitus, and an underlying health condition of the patient (such as asthma, COPD, failure to thrive, underlying unknown lung diseases such as AIDS-related illnesses or Interstitial Lung Diseases, start lower and go slower). In the preferred embodiment, the volumetric dosage of oxygen-ozone mixture (Oxygen Ozone Regenerative Therapies, OORT) is increased by approximately 10 mL in each successive treatment session. However, the dosage escalation can be reduced or completely removed if the patient is experiencing procedural complications such as Herxheimer reaction, expresses discomfort, cough, chest tightness, facial flush, or vein irritation. Vein discomfort may be mitigated by slowing down the infusion rate. Thus, the specified volume for the dosage escalation should typically range between 0 mL to 10 mL, but no more. In addition, the administrator must also make sure to prevent any infiltration.

During an initial session for the plurality of treatment sessions, the volumetric dosage of oxygen-ozone mixture (Oxygen Ozone Regenerative Therapies, OORT) is within a preferred range of 10 to 20 mL for a 90-or-more pound (lb.) person. The exact volumetric dosage of oxygen-ozone mixture (Oxygen Ozone Regenerative Therapies, OORT) for the initial session is also proportionately depended upon a weight, a habitus, and an underlying health condition of the patient and is preferably determined by the administrator. Consequently, a lighter patient would require a dosage closer to 10 mL, and a heavier patient would require a dosage closer to 20 mL. However, the present invention allows for an even-lower volumetric dosage of oxygen-ozone mixture (Oxygen Ozone Regenerative Therapies, OORT) of 2.5 mL if the patient's weight is proportionately low as well.

From a tenth session to a twelfth session for the plurality of treatment sessions, the volumetric dosage of oxygen-ozone mixture (Oxygen Ozone Regenerative Therapies, OORT) is about 55 mL for an approximately 90-or-more lb. person. Likewise, the exact volumetric dosage of oxygen-ozone mixture (Oxygen Ozone Regenerative Therapies, OORT) for these later sessions is proportionately depended upon a weight, a habitus, and an underlying health condition of the patient and is preferably determined by the administrator.

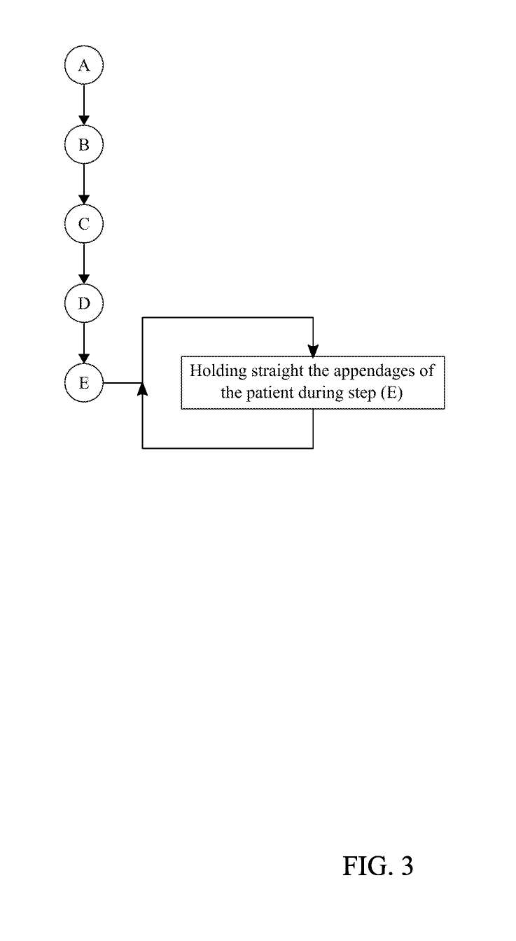

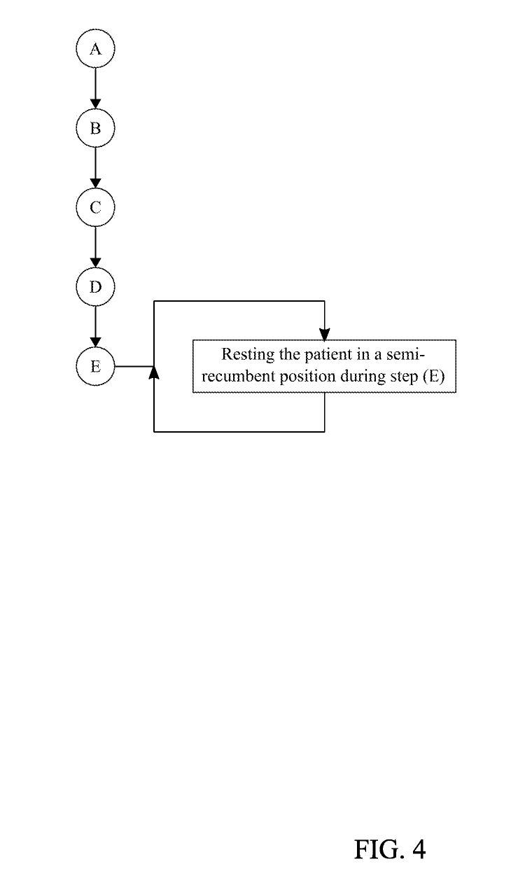

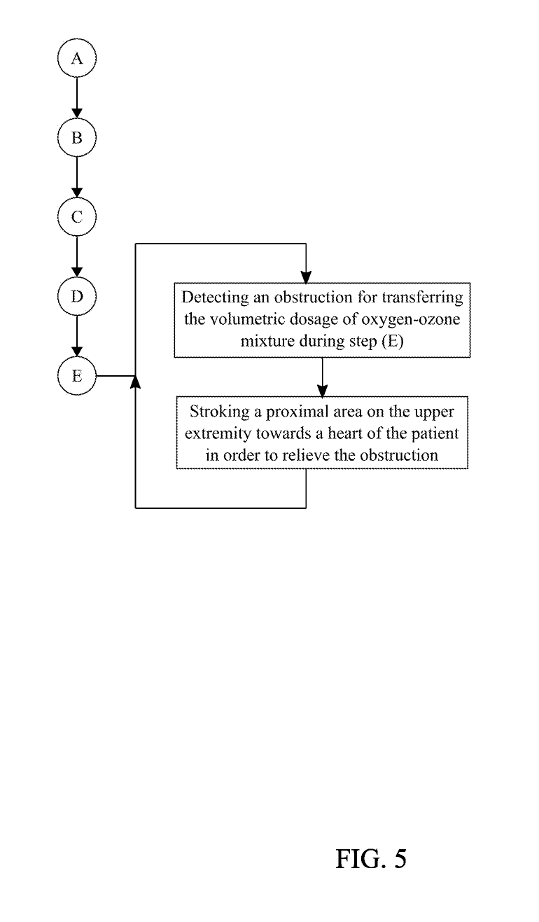

The present invention also allows for different actions to improve the efficiency of Step E. In reference to FIG. 3, one such action is to have the patient straighten their appendages during Step E, which prevents an obstruction from occurring as the volumetric dosage of oxygen-ozone mixture (Oxygen Ozone Regenerative Therapies, OORT) transfers into the accessible peripheral vein. This means that the patient's appendages should be outstretched and not bent. In reference to FIG. 4, another such action is to have the patient resting in a semi-recumbent position in order to improve the efficiency of Step E. In addition, if an obstruction or resistance to pushing of gaseous mixture is encountered for transferring the volumetric dosage of oxygen-ozone composition is detected during Step E, then another such action that could be taken by the administrator is to stroke the proximal area on the upper extremity towards the heart of the patient, which is shown in FIG. 5. This stroking or “milking” movement by the administrator should relieve the obstruction felt by the patient. In reference to FIG. 6, another such action is to have the patient do a weight-lifting exercise to improve circulation around the cannulation area for days prior to and on day of procedure and on the day of the procedure. Another such action is to have the patient drink plenty of liquids and be hydrated before a treatment session.

The present invention also allows the overall process to be simultaneously repeated between the patient and the administrator. Thus, the administrator is able to simultaneously execute a first iteration of Steps A through E with a first syringe and a second iteration of Steps A through E with a second syringe. This allows the administrator to double the volumetric dosage of oxygen-ozone mixture (Oxygen Ozone Regenerative Therapies, OORT) during a single treatment session, while first clamping (with taped jaws) the tubing at the distal end of the intravenous tubing, to prevent air entry, while switching out syringes. Special safety precautions need to be taken by the administrator for the patient while using two syringes. For the plurality of treatment sessions, the simultaneous execution of the first iteration and the second iteration is done during the later treatment sessions. In the preferred embodiment, the simultaneous execution of the first iteration and the second iteration allows the volumetric dosage of oxygen-ozone mixture (Oxygen Ozone Regenerative Therapies, OORT) to range between 110 mL to 120 mL.

Moreover, the infusion device is removed after the complete infusion of the desired dosage, whereupon it is desired that the patient compresses the cannulated vein for at least 5 minutes without bending the body part containing the vein. After the treatment, the administrator observes the patient for at least 10 minutes before allowing the patient to leave. The administrator must note symptoms such as cough, chest tightness, or anything unexpected. It is desired that the administrator leaves 5 mL of ozone in the syringe to keep the syringe from sucking in residual bodily fluids of the patient (and subsequently, when withdrawing needle-cannula out, push that 5 ml out, after withdrawing cannula, to sterilize needle and syringe contents and barrels to avoid any risk of disease transmission). The syringe should be reusable until a black ring inside the plunger become visible, or if the plunger gets too stiff to move it, or if the graduation markings fade and become obscure. It is also preferable that the administrator switch veins and arms being cannulated on a particular treatment session to allow the veins to recuperate and avoid repeated trauma to the same veins.

In regard to the side effects associated to the present invention, a cerebral paresis has been observed by the inventor in 1 out of 10,000 cases. A feeling of paresis may begin on one side of the patient's body within the first 30 minutes after the push starts. The feeling of paresis lasts from 2 to 30 minutes after the treatment and has not led to any residual effects. Cerebral paresis is only a sensation of weakness, not a related to actual physiological damage.

These side effects have never happened with the initial treatment session or during the actual treatment. If the patient has experienced cough or chest tightness, the dosage is not increased until he or she has the same dose again without any such experience.

In the rare event that distressing chest symptoms occur for the patient, oxygen is given at 3 litres per minute via the nasal cannula to speed the resolution of the chest discomfort. The patient is not discharged until he/she is well past the symptoms, and the patient is observed for 5 to 10 minutes after the symptoms disappear before patient leaves, whereupon the patient is breathing O₂ for the whole time, which may take 30 minutes to an hour to settle down or resolve entirely.

For vein irritation, warm compresses every 10 minutes, Traumeel cream, and as a last resort, ibuprofen is applied to the cannulation area. To prevent vein irritation, the administrator makes sure that the patient is taking vitamin C to gut tolerance every 2 hours, 5 to 6 times a day. Also, the patient is made to drink 1 gallon of water everyday (if not contraindicated) and take at least 3 ounces of probiotic bio-juice daily to replenish bacteria in intestinal wall.

Although the invention has been explained in relation to its preferred embodiment, it is to be understood that many other possible modifications and variations can be made without departing from the spirit and scope of the invention as hereinafter claimed. A Herxheimer reaction may also present as feverishness, joint aches and pains, and whole body pain as in a flu-like syndrome—this can last 1-2 days, and maybe addressed by rest and hydration.

What is claimed is:

1. A method of administering an intravenous oxygen-ozone mixture (Oxygen Ozone Regenerative Therapies, OORT), the method comprising the steps of:
(A) providing a syringe, an infusion device and a tourniquet;
(B) identifying an accessible peripheral vein on an upper extremity of a human;
(C) preparing a volumetric dosage of the oxygen-ozone mixture (Oxygen Ozone Regenerative Therapies, OORT) with the syringe, the volumetric dosage of the oxygen-ozone mixture (Oxygen Ozone Regenerative Therapies, OORT) being 96% oxygen and 4% ozone (or a concentration of 55 mcg of ozone gas per ml of oxygen-ozone gaseous mixture);
(D) applying the tourniquet to a cannulation area on the upper extremity and inserting the infusion device into the accessible peripheral vein; and
(E) transferring the volumetric dosage of the oxygen-ozone mixture (Oxygen Ozone Regenerative Therapies, OORT) from the syringe, through the infusion device, and into the accessible peripheral vein at a specified infusion rate by releasing the tourniquet from the cannulation area after a witnessed flash of blood.

2. The method as claimed in claim 1 comprising the steps of:
repeating steps (B) through (E) as a plurality of treatment sessions;
incrementally increasing the volumetric dosage of the oxygen-ozone mixture (Oxygen Ozone Regenerative Therapies, OORT) by a specified volume during each treatment session of the plurality of treatment sessions.

3. The method as claimed in claim 2, wherein the volumetric dosage of the oxygen-ozone mixture (Oxygen Ozone Regenerative Therapies, OORT) during an initial session is within a range of 10 to 20 milliliters (mL) and proportionately depends upon a weight and a habitus of the human patient, and wherein the initial session is from the plurality of treatment sessions.

4. The method as claimed in claim 2, wherein the specified volume is 10 mL.

5. The method as claimed in claim 2, wherein the volumetric dosage of the oxygen-ozone mixture (Oxygen Ozone Regenerative Therapies, OORT) from a tenth session to a twelfth session is 55 mL and proportionately depends upon a weight and a habitus of the human patient, and wherein the tenth session to the twelfth session is from the plurality of treatment sessions.

6. The method as claimed in claim 1, wherein the infusion device is a 27-gauge infusion set and is in fluid communication with the syringe.

7. The method as claimed in claim 1, wherein the specified infusion rate is 1 mL every 5 to 15 seconds.

8. The method as claimed in claim 1, wherein the specified infusion rate is adjusted to be proportionately slower for a smaller diameter of the accessible peripheral vein.

9. The method as claimed in claim 1, wherein appendages of the human patient are held straight during step (E).

10. The method as claimed in claim 1, wherein the human patient is resting in a semi-recumbent position, with outstretched limbs, during step (E).

11. The method as claimed in claim 1 comprising the steps of:
detecting an obstruction by increased resistance displayed towards the intravenous pushing of the gaseous mixture for transferring the volumetric dosage of the oxygen-ozone mixture (Oxygen Ozone Regenerative Therapies, OORT) during step (E); and
stroking a proximal area on the upper extremity towards a heart of the human patient in order to relieve the obstruction.

12. The method as claimed in claim 1 comprising the step of:
simultaneously executing a first iteration of steps (A) through (E) with the syringe and a second iteration of steps (A) through (E) with another syringe in order to double the volumetric dosage of the oxygen-ozone mixture (Oxygen Ozone Regenerative Therapies, OORT) for a treatment session, after first clamping the tubing of the IV catheter, while switching out an empty syringe for a syringe full of above gaseous mixture, and unclamping this tubing, as soon as the full syringe is connected.

13. The method as claimed in claim 1, wherein the volumetric dosage of the oxygen-ozone mixture (Oxygen Ozone Regenerative Therapies, OORT) doubles to a range between 110 mL to 120 mL over 30 treatment sessions extendable to over a 6-7 month period, for a more protracted retention period of therapeutic effects of the gaseous mixture.

14. The method as claimed in claim 1 comprising the step of:
prompting the human patient to initiate a weight-lifting exercise in order to improve blood circulation around the cannulation area.
